(19) United States
(12) Patent Application Publication
(10) Pub. No.: US 2026/0000569 A1
(43) Pub. Date: Jan. 1, 2026

(54) Postoperative Therapeutic Pressure System

(71) Applicant: Steven James Bates, Palo Alto, CA (US)

(72) Inventor: Steven James Bates, Palo Alto, CA (US)

(21) Appl. No.: 18/757,174

(22) Filed: Jun. 27, 2024

(51) Int. Cl. A61H 9/00; A61F 13/05

(52) U.S. Cl. CPC A61H 9/0092; A61F 13/05; A61H 9/0007; A61H 2201/169; A61H 2201/5071; A61H 2209/00

(57) ABSTRACT

Various aspects of the disclosure generally relate to a postoperative therapeutic pressure system that may be worn by patients after undergoing surgery, including body contouring cosmetic procedures. The system may include an inflatable bladder connected to a pump, the inflatable bladder held near the patient with one or more wraps, or other postoperative garments. The bladder may be inflated to a series of different pressures and the pressure held for a time period. After releasing the pressure, a rest period may follow.

### BACKGROUND

### Field of the Disclosure

Aspects of the disclosure address compression therapy for reducing post-operative edema, distinct from a disease state such as lymphedema. Post-operative edema may result from plastic surgery, both cosmetic and reconstructive.

### Description of Related Art

Cosmetic and reconstructive procedures may result in soft tissues becoming swollen with fluid due to surgical trauma. Normally, the lymphatic system removes extracellular fluid (buildup of fluid in the tissues-edema) through a rhythmic mechanical pumping action exerted by the normal contractions of the musculoskeletal system. This drainage system is disrupted by surgical trauma to the tissues causing leakage of fluid out of cells and capillaries producing the buildup of tissue edema. The pooling of this edema fluid has negative effects on the outcome of surgical procedures including increased pain and discomfort, stiffness, prolonged recovery, and undesirable cosmetic appearance.

Compression therapy (CT), is the selective external compression of a portion of the body using wraps, stockings, inflatable cuffs and bandages. CT can be either passive compression using elastic or inelastic bandages or multiple layers of bandages (no external energy applied) or active, where an external energy source augments a compressive force applied to body part(s). CT can help in pushing interstitial fluid back into the vascular and lymphatic systems, reducing edema and promoting its reabsorption. Moderate compression is strong enough to prevent blood from pooling in the veins but not so strong that it causes venous congestion. Venous congestion can occur if the pressure is too high, as it may obstruct venous outflow.

Examples of some commercially available compression bandages include those made by 3M, BSN Medical, Convatec, Derma Sciences, Hartman group, Kendall/Covidien, Lohmann and Rauscher, Medline Industries, and Smith and Nephew. The compressive force of compression bandages is achieved in the application or wrapping of the bandage by a caregiver. The consistency of compression is dependent on the skill of the caregiver applying the bandage and feedback typically occurs for the caregiver by the amount of “pull” or force exerted during bandage application. The patient wears the bandage until it loses its compliance or becomes dirty.

Typically, compression garments and binders are worn after body contouring procedures in an attempt to decrease the amount of swelling that occurs in the postoperative setting. Garments and binders are merely “fitted” to the patient after surgery and the practitioner must rely on the inherent strength and elasticity of the garment to hopefully apply pressure within a helpful (and not harmful) range. Too loose a garment will allow for increased edema and swelling, leading to poor clinical outcomes and possible disease states such as impaired wound healing and chronic lymphedema. Too tight a garment can cause venous congestion and impede arterial flow, both of which can lead to adverse outcomes. As tissues inevitably swell, circumferential compression garments can become tighter, thereby constricting the underlying and surrounding tissues like a tourniquet. This pattern of swelling and then increased circumferential tightening further prevents the clearance of edema fluid due to mechanical obstruction of the lymphatic system leading to both venous congestion and tissue ischemia from decreased arterial flow.

Compression therapy is helpful in treating or preventing edema. The significant deficiencies that compression garments and therapy suffer from are unknown/inconsistent pressure application, physical discomfort, variable pressures due to physical activity and volumetric changes of soft tissue after surgery. Another issue is the need for financial investment in garments of different sizes or compression, to manage changing pressure needs. These deficiencies result in either non-compliance or improper use, which can over time cause postoperative edema to result in a disease state, for example wound healing complications and chronic lymphedema. Additionally of concern to patients receiving cosmetic surgery are unwanted cosmetic outcomes

What is needed is a postoperative therapeutic pressure system that addresses these deficiencies and is directed toward pre-disease states of edema and improved cosmetic outcomes.

### SUMMARY

The systems, methods and devices of this disclosure each have several innovative aspects, no single one of which is solely responsible for the desirable attributes disclosed herein. In one aspect a postoperative therapeutic pressure system may include an inflatable bladder with the bladder pressure-limited to a peak pressure of 25 mmHg and having an uninflated surface area no greater than 260 square inches. A pump may be coupled to the bladder, with the pump configured to maintain a plurality of positive, non-zero pressures within the bladder. A pressure board may be adjacent to the bladder with the board resistant to deformation. A pouch may be configured to receive and secure the pressure board and the bladder, with the bladder further having two sides, the pressure board immediately adjacent to one of the two sides. A smoothing layer may be immediately adjacent to the bladder on the side opposite the pressure board.

In one aspect, a pressure sensor in the pump may be configured to provide the pressure for the bladder, with the pump further configured to maintain the pressure-limit in the bladder.

In one aspect, the pump may be further configured to increase the peak pressure after a safety period.

In one aspect, the safety period may be 7 days.

In one aspect, the pump may be further configured to increase the peak pressure to 50 mmHg after 7 days and configured to increase the peak pressure to 75 mmHg after 14 days.

In one aspect, the pump may be further configured, with the bladder in a depressurized state, to hold for a first time period, to increase pressure in the bladder to a pressure level and pause for a second time period, and release pressure in the bladder.

In one aspect, the pressure board may further comprise a moldable, foam-aluminum composite.

In one aspect, the pump may be further configured with a first intensity level, a second intensity level and a third intensity level, the first intensity level having a pressure level from 20 to 30 mmHg, the second intensity level having a pressure level from 40 to 60 mmHg, the third intensity level having a pressure level from 70 to 80 mmHg. The pump may be further configured to maintain bladder pressure at or under the peak pressure.

In one aspect, the first time period is from 10 to 20 seconds, with the pump configured with a first mode, a second mode and a third mode. The first mode may have a second time period from 20 to 40 seconds, the second mode having a second time period from 50 to 70 seconds, and the third mode having a second time period from 80 to 120 seconds.

In one aspect, the pressure board, the bladder and the smoothing layer may have the same shape.

In one aspect a multi-panel, stretchable and compressive wrap may be coupled to the pouch, the pouch may be configured to be removably affixed to the wrap.

In one aspect, the pouch may be neoprene rubber with a plurality of layers forming pockets, with the pockets having VELCRO enclosures. The smoothing layer may be one of the layers of the pouch.

In one aspect, the pouch may be neoprene rubber with a plurality of pockets, with the pockets having VELCRO enclosures. The smoothing layer may be foam and immediately adjacent to the bladder, with both the bladder and the smoothing layer in one of the plurality of pockets.

In one aspect, the pressure board may be in one of the plurality of pockets not containing the bladder.

In one aspect, a tube may be coupled to the pump with a right-angle connector connected to the bladder. A reinforced sleeve may be between the tube and the right-angle connector.

There is a method for providing postoperative lymphatic massage in a system having an inflatable bladder. The bladder may be pressure-limited to a peak pressure of 25 mmHg and have an uninflated surface area no greater than 260 square inches. There may be a pump coupled to the bladder with the pump configured to maintain a plurality of positive, non-zero pressures within the bladder. A pressure board may be adjacent to the bladder, with the board being formable and resistant to deformation. A smoothing layer may be coupled to and adjacent to the bladder. A multi-panel, stretchable and compressive wrap may be coupled to the bladder, with the bladder configured to be removably affixed to the wrap. The method may include applying the bladder to a patient in a postoperative setting, securing the bladder to the patient with the wrap. And activating the pump to affect a pressure increase in the bladder to a pressure level with a pause for a second time period, and a pressure release in the bladder with a pause, following release, for a first time period.

In one aspect, the method may include fitting a compression garment to the patient, there being at least some overlap between the garment and the bladder.

In one aspect, the method may have a pressure level within the range of 5 to 25 mmHg, the first time period from 10 to 20 seconds, and the second time period from 30 to 90 seconds.

In one aspect, the method may include forming the board.

In one aspect, the method may include applying a second bladder to the patient in the postoperative setting, with the second bladder in a different location on the patient than the bladder; and connecting the second bladder to the pump, the pump to affect a pressure increase in the second bladder.

The foregoing has outlined rather broadly the gestures and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of this disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF THE DRAWINGS

So that the above-recited features of the disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 is a perspective view illustrating one aspect of a postoperative therapeutic pressure system positioned for application to a patient.

FIG. 2 is a perspective view illustrating one aspect of a postoperative therapeutic pressure system applied to a patient and secured with a double wrap.

FIG. 3 is a perspective view illustrating one aspect of a postoperative therapeutic pressure system applied to a patient after a surgical procedure.

FIG. 4 is a perspective view illustrating components of one aspect of a postoperative therapeutic pressure system.

FIG. 5 is a plan cutaway view illustrating one aspect of a postoperative therapeutic pressure system.

FIG. 6 is a schematic diagram of one aspect of a pump for a postoperative therapeutic pressure system.

FIG. 7 is a block diagram illustrating a peak pressure limit for a bladder pressure in a postoperative therapeutic pressure system.

FIG. 8 is a block diagram illustrating a pressure limit for a safety period in a postoperative therapeutic pressure system.

FIG. 9 is a block diagram illustrating an increase in allowed peak pressure in a postoperative therapeutic pressure system.

FIG. 10 is a block diagram illustrating rhythmic compressions in a postoperative therapeutic pressure system.

FIG. 11 is a block diagram illustrating modes and intensity levels for one aspect of a pump in a postoperative therapeutic pressure system.

FIG. 12 is a block diagram illustrating one method for providing postoperative lymphatic massage with a postoperative therapeutic pressure system.

### DETAILED DESCRIPTION

Various aspects of the disclosure are described more fully herein with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based at least in part on the teachings herein, one skilled in the art should appreciate that the scope of the disclosure is intended to cover any aspect of the disclosure disclosed herein, whether implemented independently of or combined with any other aspect of the disclosure. For example, an apparatus may be implemented, or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. Any aspect of the disclosure may be embodied by one or more elements of a claim. Dashed lines in the Figures may be used to indicate structure behind or hidden from plan or perspective view.

In one aspect, a postoperative therapeutic pressure system may be worn by patients after undergoing plastic surgery, including body contouring cosmetic procedures such as 360 liposuction, Brazilian butt lift (BBL), abdominoplasty or jawline and face contouring procedures. The system may include an inflatable bladder connected to a pump, the inflatable bladder held near the patient with one or more wraps, or other postoperative garments. Against the bladder and distal to the patient there may be a shapeable pressure board to assist with directing pressure to the patient. The inflatable bladder is rhythmically inflated and deflated to apply varying levels of pressure to the operative site. The pressure system may be targeted at areas where swelling persists after surgery (for example, the abdomen, thigh, calf, arm, lower back, chin, etc.).

Rhythmic compression externally exerted on the soft tissues can mimic the normal pumping action of the lymphatic system. Sequential compression of soft tissue up to a peak pressure (identified throughout this description with millimeters of mercury-mmHg) can reduce the likelihood of lymphatic stasis and improve clearance of edema fluid. The peak pressure is safe and effective for a given stage of healing as identified by the passage of time from the surgery.

Rhythmic compression is also the mechanism by which lymphatic massage reduces edema after body contouring procedures. The postoperative therapeutic pressure system uses the concept of Rhythmic Compression Therapy (RCT) to mechanically “pump” postoperative edema out of the skin and soft tissue and into the lymphatic system for clearance. Benefits of the system may include positioning accuracy of the bladder to the area most in need of the therapy, programmed modes and intensity levels varying according to how much time has passed since the surgery, comfort of the patient, and individual swelling response (which are not possible to predict) to surgery, which is managed by a safety period and peak pressures. Another benefit of the system is that patients can tolerate higher peak pressure when that pressure is applied rhythmically by a postoperative therapeutic pressure system, than would be tolerated by static application of that pressure. Additionally, the system is more economical and more accurate than in-house RCT (by a massage therapist, for example), and may be worn and activated as often as needed by the patient. The safety period is a time during which wound healing occurs and tissues are at greater risk of ischemic injury or venous congestion with the application of higher pressure. During the safety period, limiting peak pressure also helps with pain and therefore patient compliance may be higher.

One of skill in the art will recognize that “lymphatic massage” is a term applied to care provided after plastic surgery, whereas RCT is a term applied to work done by a massage therapist. The postoperative therapeutic pressure system may be applied in either or both situations (as a substitute for lymphatic massage or RCT) and those terms are used interchangeably herein. There is no claim made that these are identical treatments, or that the mechanism by which the postoperative therapeutic pressure system works is the same.

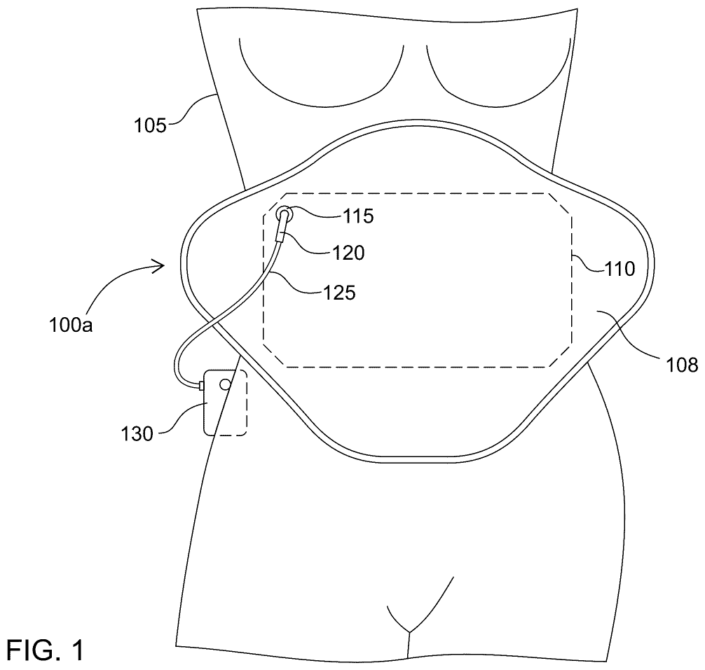

FIG. 1 is a perspective view illustrating one aspect of a postoperative therapeutic pressure system positioned for application to a patient. Postoperative therapeutic pressure system 100a (also see postoperative therapeutic pressure systems 100b, 100c and 100d, collectively referred to as postoperative therapeutic pressure systems 100, in later Figures) is illustrated in front of patient 105. In one aspect, postoperative therapeutic pressure system 100a (system 100a) includes, in part, pouch 108 containing inflatable bladder 110. Inflatable bladder 110 includes an inflation port for receiving air, the inflation port including right-angle connector 115 extending from bladder 110 from out of a hole in pouch 108. In one aspect, right-angle connector 115 may connect to reinforced sleeve 120 and then to tube 125. Tube 125 is a fluid conduit between inflatable bladder 110 and pump 130. Pump 130 may have an output (not identified in FIG. 1) connected to tube 125 and other components, see FIG. 8. Other components of system 100a may include a pressure board (in one aspect, optional, in one aspect, included at initial application or at a later time), pouch and smoothing layer, not illustrated in FIG. 1 for simplicity. Pump 130 may be small and portable and attachable to the wrap, binding or garment so that patients have freedom of movement. Pouch 108 may be neoprene, for example.

In one aspect, bladder 110 may be rectangular with dimensions approximately 11″×12″, or have a total uninflated (external) surface area of approximately 260 square inches (accounting for both external sides of bladder 110). In one aspect, systems 100 may have a bladder with a total surface area of 260 square inches as a maximum for therapeutic affect from rhythmic compressions. Other dimensions for a rectangular shape may be anywhere from 5″ on a side to 11″ on that side, and from 6″ on the adjacent side to 12″ on that side. Exemplary sizes include 6″×10″, which may be used for treatment of postoperative abdominal edema, and 2″×5″, which may be used for a facelift, or neck liposuction, for example. In one aspect, an external surface area for bladder 110 may be between 100 and 140 square inches. Bladder 110 may approximate shapes other than a rectangle, for example square, circle, triangle, donut, hexagon, or shaped like pouch 108. Bladder 110 may be made from a thermoplastic polyurethane elastomer (TPU) which is closed by heat sealing, or with other materials and methods suitable for inflation by a fluid mechanism, for example air.

In one aspect, the inflation port of bladder 110 may be a straight-in connector, perpendicular to the inflated surface of bladder 110, or an angled connector, for example a 45-degree angle connector, or right-angle connector 115. In one aspect, the inflation port may pass through a hole in pouch 108, dedicated to the inflation port. In one aspect, pouch 108 does not have a dedicated hole for the inflation port, so tube 125 would exit from, for example, the same place into which bladder 110 is inserted into pouch 108.

In one aspect, the inflation port is made from a suitable material, for example TPU or polyvinyl chloride (PVC). The inflation port, for example right-angle connector 115, may connect to reinforced sleeve 120, which may be, for example, a high pressure tube with ⅛ inch inner diameter (ID) and ¾ inch outer diameter (OD). Reinforced sleeve 120 may connect to tube 125, which may be a soft silicon with ⅛ inch ID and ⅜ inch OD, for example.

After a surgery involving, for example, the abdomen, and prior to being discharged, system 100a may be positioned over the area where swelling is expected and secured to patient 105 with flexible wraps or garments (not illustrated in FIG. 1). A wrap may be wound 1, 2, 3, 4, 5 or more times around patient 105. One advantage of system 100a is that establishing a “correct” pressure with the wrap isn't as important compared to prior techniques because system 100a is pressure regulated with pump 130. One advantage of systems 100 over traditional compression garments is that a desired pressure may be applied directly to a part of the patient's body benefiting from that pressure, as opposed to a circumferential pressure that is not sufficiently targeted to the areas with edema. If applied shortly after surgery (for example in a postoperative setting), bladder 110 may be pressure-limited to a peak pressure. In one aspect, 25 mmHg of pressure may be the peak pressure allowed by pump 130 for a safety period, for example 7 days post-surgery. In one aspect, peak pressure following surgery may be different than 25 mmHg, for the time period immediately following surgery, according to best practice as it evolves within the medical community. For example, if 20 mmHg for 60 hours is discovered to have better results, then bladder 110 may be pressure limited to a peak pressure of 20 mmHg for the safety period of 60 hours. Other safety periods may be from 24 to 168 hours. The safety period and other time periods as measured by the pump may be measured from the time of surgery. One of skill in the art will recognize that other benchmarks for time may be followed to achieve the same result, for example if a patient has a follow-up consultation with a care provider scheduled 7 days after surgery, and system 100a is applied at that time, a safety period (in this example, of approximately 7 days) may have already elapsed (post-surgery) and a peak pressure higher than, for example 25 mmHg, may be set for initial treatment. In this example, peak pressure may start at 50 mmHg.

Pump 130 is programmed to allow the peak pressure to increase over time. In one aspect, peak pressure may be allowed to increase after the safety period (for example, 7 days) to a higher pressure. After another time period, peak pressure may be increased again. In one example, peak pressure may be increased to 50 mmHg after 7 days. In one example, peak pressure may be increased to 75 mmHg after 14 days. In one aspect, in a first use of system 100a for patient 105 when the patient is past the safety period, the peak pressure may start at 50 mmHg and then increase to 70 mmHg at the appropriate time, post-surgery.

In one aspect, pump 130 includes a pressure sensor (not illustrated in FIG. 1, see FIG. 8). The pressure sensor enables pump 130 to stay within the pressure limit defined by the peak pressure. In operation, pump 130 increases pressure in bladder 110 through the fluid connection provided by tube 125. Bladder 110 is pressure limited by pump 130 to the peak pressure and in operation will go from depressurized up to, but not exceeding, peak pressure. One advantage of system 100a is that over time, patient 105 may experience swelling, resulting in pump 130 using less air to reach peak pressure. In one aspect, if swelling decreases, pump 130 may use more air to reach peak pressure (in both examples, peak pressure not changing). Additionally, if the wraps or garments loosen, for example, pump 130 may use more air to reach the same peak pressure than previously used.

Continuing with operation, pump 130 may increase pressure in bladder 110 over a time period, up to the peak pressure, and hold the pressure for another time period. Following that, pump 130 may release the pressure and repeat the sequence. See FIG. 13 for additional modes.

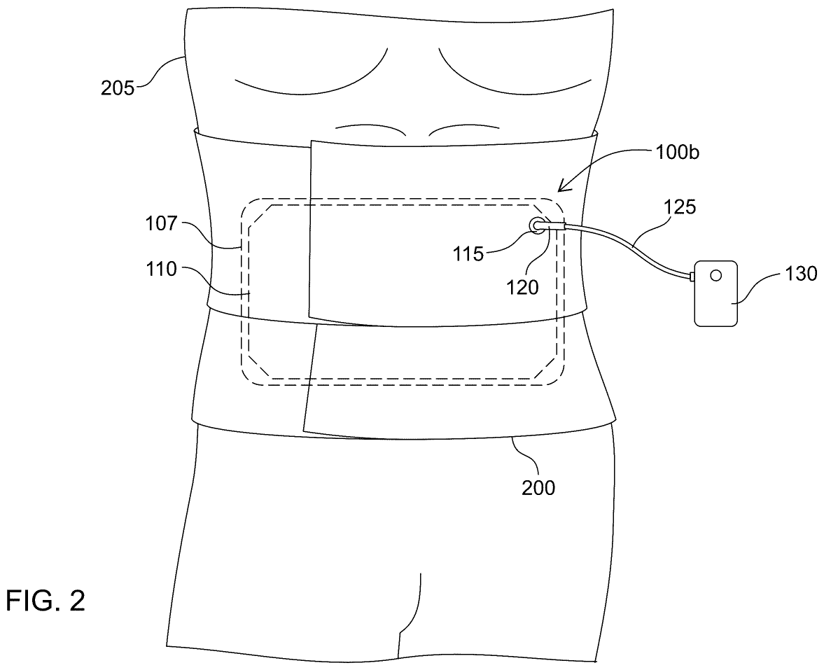

FIG. 2 is a perspective view illustrating one aspect of a postoperative therapeutic pressure system applied to a patient and secured with a double wrap. In one aspect, postoperative therapeutic pressure system 100b (system 100b) is illustrated with wrap 200 affixed to patient 205. In one aspect, patient 205 may have had surgery involving the abdomen, and prior to being discharged, system 100b may be positioned over the area where swelling is expected and secured to patient 205 with a wrap (for example, a compression garment). After the safety period has passed and higher peak pressure is available in system 100b, wrap 200, which may be a double panel “binder,” or a multi-panel stretchable and compressive fabric, may be applied. In one aspect, wrap 200 may overlap compression garments (not illustrated in FIG. 2). In one aspect, wrap 200 may replace compression garments. Wrap 200, as a double binder, may be more effective than compression garments for system 100b achieving peak pressure above 25 mmHg. In one aspect wrap 200 includes two panels, each about 9″ in height, with one panel longer than the other. Various lengths of wraps may be made to fit patients of different circumferences. Wrap 200 may fit around patient 205 and secure to itself with clips, clasps, VELCRO, or other fastening mechanisms. In one aspect, multi-panel stretchable and compressive wrap 200 may be made from a combination of polyester and LYCRA. In one aspect, the polyester and LYCRA blend is 82%/18%. In one aspect, other materials may be used and other ratios incorporated into the wrap.

System 100b may include similar or the same components as system 100a and may operate in a similar fashion. System 100b includes pouch 107, with a rectangular shape and may have dimensions of 5-12 inches wide and 2-11 inches high. In one aspect, pouch 107 is identical to pouch 108 except in size and shape. In one aspect, pouch 107 has dimensions of 2″×5″, 8″×12″, 6″×11″ or 8″×11″. Pouch 107 may have fasteners for removably fixing or securing to a binder or wrap 200, such as VELCRO. Pouch 107 is behind wrap 200, such that pouch 107 is between wrap 200 and patient 205.

Within pouch 107 is bladder 110. Bladder 110 may connect through connector 115 to reinforced sleeve 120 and tube 125 to pump 130.

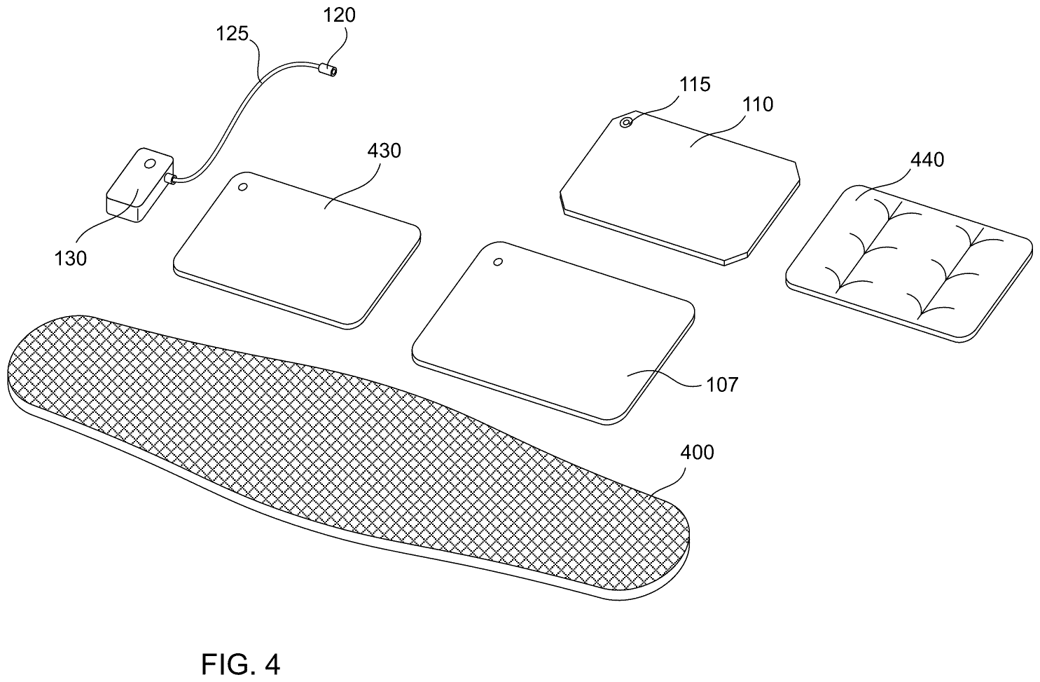

Not illustrated in FIG. 2 for simplicity, and one aspect of system 100b, are a smoothing layer and a pressure board (see FIG. 4 for examples of both).

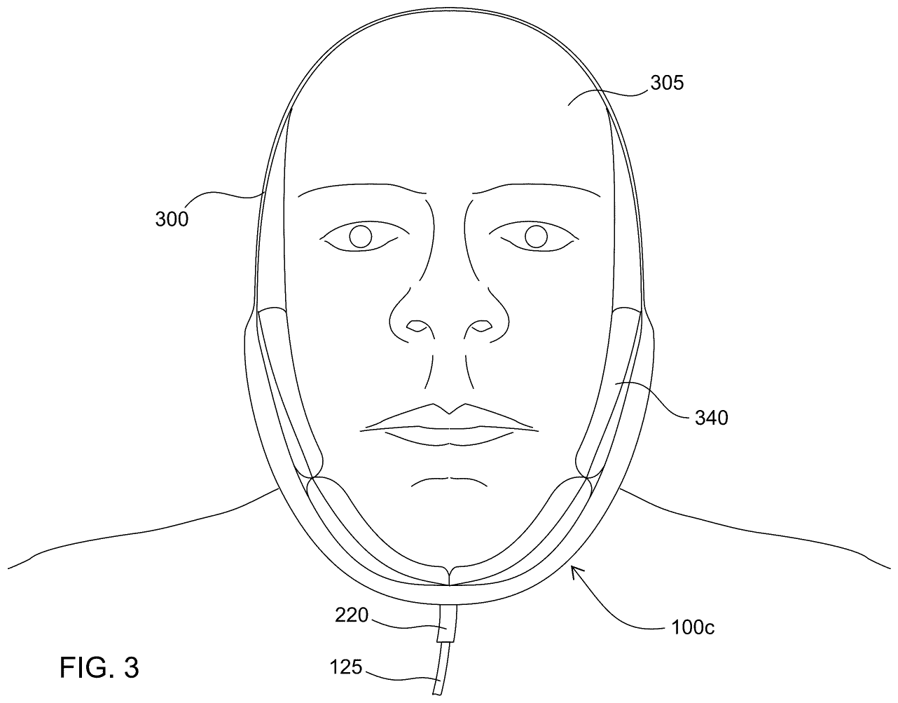

FIG. 3 is a perspective view illustrating one aspect of a postoperative therapeutic pressure system applied to a patient after a surgical procedure. In one aspect, postoperative therapeutic pressure system 100c (system 100c) is illustrated with wrap 300 affixed to patient 305. In one aspect, patient 305 may have had surgery involving the chin or neck, for example a facelift, necklift, cervicoplasty and neck liposuction procedure. Prior to being discharged, system 100c may be positioned over the area where swelling is expected and secured to patient 305 with wrap 300, which may be gauze, an elastic band, or a stretchable and compressive fabric. Various lengths of wraps may be made to fit patients with different head sizes. Wrap 300 may fit around patient 305 and secure to itself with clips, clasps, VELCRO, or other fastening mechanisms.

System 100c may include similar or the same components as systems 100a and 100b and may operate in a similar fashion. System 100c includes a bladder (not illustrated in FIG. 3), with a rectangular shape and may have dimensions of approximately 2″×5″. The bladder may include the functionality of bladder 110. The bladder may be within a pouch (not illustrated in FIG. 3), and the pouch may have fasteners for removably fixing or securing to a binder or wrap 300, such as VELCRO. The pouch is secured by wrap 300, such that the pouch is between wrap 300 and patient 305. The bladder may connect through a straight-line connector (not illustrated in FIG. 3) to reinforced sleeve 220 and tube 125 to the pump (not illustrated in FIG. 3).

One aspect of system 100c is smoothing layer 340. Smoothing layer 340 isolates patient 305 from the bladder so that creases, folds and irregularities in the bladder at various pressures do not imprint on the skin of patient 305. In one aspect, smoothing layer 340 may have similar dimensions to the pouch. Smoothing layer 340 may be made of a soft, smooth foam. In one aspect, a smoothing layer may be an insertable layer in a pouch, which may secure the bladder and a pressure board (see FIG. 4 for examples of both). In one aspect, a smoothing layer may be integral to the pouch (i.e. an inseparable part of the pouch). A pressure board is not illustrated in FIG. 3 but may be a component of system 100c. In one aspect, the straight-line connector may be similar to connector 115 (refer to the description for FIG. 1), except the straight-line connector emerges from the bladder perpendicular to the surface of the bladder. In one aspect, the straight-line connector may be replaced by a right-angle connector.

FIG. 4 is a plan view illustrating components of one aspect of a postoperative therapeutic pressure system. In one aspect, postoperative therapeutic pressure system 100d (system 100d) is illustrated, and in addition to system 100d is wrap 400. System 100d may include similar or the same components as systems 100a, 100b and 100c and may operate in a similar fashion. FIG. 4 illustrates system 100d with the inclusion of pressure board 430 and smoothing layer 440.

The separate components of system 100d may fit together as follows. In one aspect, pouch 107 facilitates the placement, organization and alignment of pressure board 430, bladder 110 and smoothing layer 440 with respect to one another and a patient. Pouch 107 may have one, two or three separate compartments. In a one-compartment pouch, pressure board 430, bladder 110 and smoothing layer 440 may all be in the one compartment. In a two-compartment pouch, pressure board 430 may be in one of the compartments, while bladder 110 and smoothing layer 440 may be in the second compartment. In one aspect, bladder 110 and pressure board 430 may share a compartment, while smoothing layer 440 may be in the second compartment. In a three-compartment pouch, bladder 110, pressure board 430 and smoothing layer 440 may each be in one of the compartments.

One of skill in the art will recognize that there are other ways to place, organize and align a bladder, pressure board and smoothing layer than with a pouch. For example, a bladder, pressure board and smoothing layer may be stacked upon each other and secured with wraps or garments, or two of the three may be in a pouch (for example a pressure board and bladder, with the smoothing layer external), or they may have external attachment features such as VELCRO on each of the bladder, pressure board and smoothing layer such that they are removably affixed to one another, or the pressure board and smoothing layer may fix to one another with the bladder in between.

Tube 125, forming a fluid connection between pump 130 and bladder 110, may be routed through a hole in pouch 107 (not illustrated in FIG. 4), either between compartments, or between compartments and the exterior of pouch 107 (through, for example, a hole in the wall of pouch 107), or may extend out the opening in pouch 107 through which bladder 107 or pressure board 430 is inserted.

In one aspect, pressure board 430 may be a semi-rigid plastic with approximate dimensions of 9″×12″. In one aspect, pressure board 430 and bladder 110 may have dimensions of 6″×10″, giving bladder 110 a total surface area of approximately 120 square inches. In one aspect, the pressure board may be formable, for example an aluminum-foam composite, similar to, for example, a SAM splint. One benefit to a formable pressure board is that it may be shaped (formed) according to the patient's needs. Forming may be done by the patient or healthcare provider by placing, for example, the pouch with the pressure board over the desired body part and shaping it to the area. In one aspect, a pressure board is optional at lower peak pressure, for example during the safety period, or at a peak pressure below 25 mmHg, or below 30 mmHg, because there may be sufficient pressure from a wrap or garment. A patient may go home with one of systems 100 without a pressure board initially. After the safety period passes, a pressure board may be added to help with higher pressure level. In one aspect, bladder 110 may be sized from 20-30% of the area of a patient's abdomen. A pressure board positioned against the bladder and opposite the patient (i.e. the bladder is between the pressure board and the patient) provides a surface against which pressure in the bladder can exert force, and therefore transfer that force to the patient. The pressure board may be semi-rigid so that it can somewhat conform to the bladder as pressure in the bladder causes the bladder to increase in volume, while still providing a sufficiently firm surface for pressure to be transferred to the patient. In one aspect, the pressure board is approximately the same size as the bladder, for example within 10%. In one aspect, the pressure board is approximately the same shape as the bladder. In one aspect, the pressure board has a shape different than the shape of the bladder. In one aspect, the pressure board has a size different than the bladder by more than 10%. As illustrated in FIG. 4, pressure board 430 may have a hole through which to route right-angle connector 115, reinforced sleeve 120 and tube 125. Although a single pressure board is referred to throughout this description, more than one pressure board may be used, for example multiple boards of differing widths.

Smoothing layer 440 isolates the skin of a patient from bladder 110 so that creases, folds and irregularities in the bladder at various pressures do not imprint on the skin of a patient and cause undesirable outcomes in their procedure. In one aspect, smoothing layer 440 may have similar dimensions to pouch 110, for example within 10%. In one aspect, the smoothing layer may have dimensions that differ from the pouch by more than 10%. In one aspect, the smoothing layer may be the same shape as the pouch. In one aspect, the smoothing layer may have a different shape than the pouch. Smoothing layer 440 may be made of a soft, smooth foam with a thickness from ¼″ to ¾″, or approximately ½″. In one aspect, the smoothing layer may be an insertable layer, as illustrated in FIG. 4. In one aspect, a smoothing layer may be integral to the pouch (i.e. an inseparable, built-in part of the pouch). Smoothing layer 440 as separate and distinct from pouch 107 is an optional aspect of system 100d. In one aspect, smoothing layer 440 is an outer layer of pouch 107 that is between bladder 110 and the patient. A smoothing layer also reduces the impact of the connector and tube with respect to the patient. In one aspect, the smoothing layer may be a gel pad that can be made warm or cool.

Assembling system 100d for use on a patient, pressure board 430 goes into one end of pouch 107, with bladder 110 and smoothing layer 440 also inside pouch 107. Bladder 110 may be between pressure board 430 and smoothing layer 440. In one aspect, right-angle connector 115 is routed through a dedicated hole in pressure board 430 and pouch 107, connecting to tube 125 and pump 130. Pouch 107 may be closed with fasteners, catches, adhesive, closures, VELCRO, etc. In one aspect, pouch 107 is not closed. In one aspect, tube 125 is routed out of pouch 107 not through a dedicated hole, rather through the hole into which bladder 110 is inserted into pouch 107. System 100d may be positioned against the patient and adjacent to the area needing lymphatic pressure therapy, with the side of pouch 107 having smoothing layer 440 against the skin of the patient.

Wrap 400, which may be gauze, an elastic band, or a stretchable and compressive fabric, may be secured against system 100d. In one aspect, wrap 400 and pouch 107 may have removably secured to one another with, for example, VELCRO, prior to placement against the patient. Wrap 400 may be used to assist in holding and placing pouch 107 in the correct position prior to securing wrap 400 around the patient.

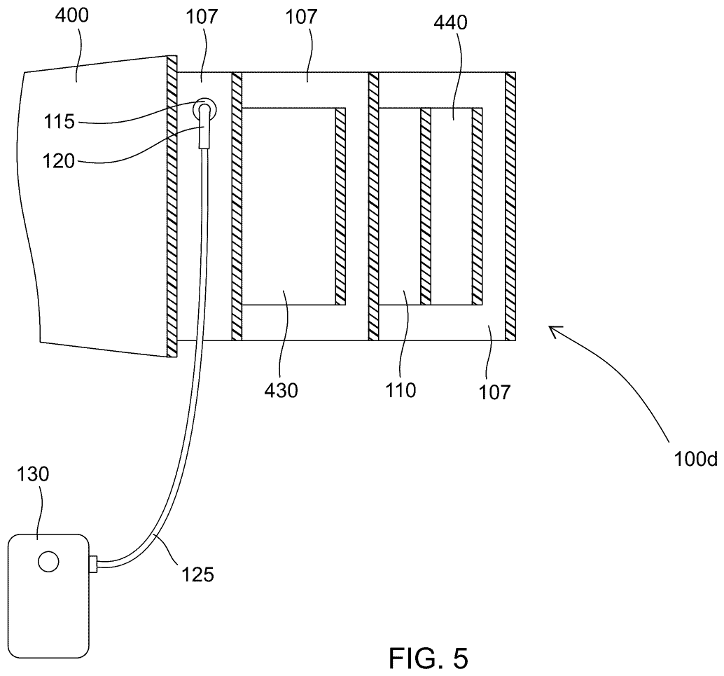

FIG. 5 is a plan cutaway view illustrating one aspect of a postoperative therapeutic pressure system. One aspect of postoperative therapeutic pressure system 100d (system 100d—also illustrated in FIG. 4), is illustrated in FIG. 5 assembled and ready for application to a patient. From the perspective of a viewer and starting with the layer closest to the viewer, the layers of system 100d are illustrated in a cut-away moving away from the viewer. The first layer and closest to the viewer is wrap 400. In one aspect, wrap 400 itself secures system 100d to the patient. In one aspect, wrap 400 represents multiple elastic compression garments that secure system 100d to the patient.

A next layer underneath wrap 400 may be pouch 107. In one aspect, pouch 107 contains pressure board 430, bladder 110 and smoothing layer 440. As previously described, pouch 107 may have a single pocket into which pressure board 430, bladder 110 and smoothing layer 440 are held. In one aspect, pouch 107 has a plurality of pockets. In one aspect, protruding through a dedicated hole is right-angle connector 115 and reinforced sleeve 120, which are connected to bladder 110. Extending from pouch 107 and under wrap 400 may be tube 125, creating a fluid connection between bladder 110 and pump 130.

A next layer after pouch 107 may be pressure board 430. As previously illustrated and described, in one aspect right-angle connector 115 and reinforced sleeve 120 extend from bladder 110 through a dedicated hole in pressure board 430.

A next layer after pressure board 430 may be pouch 107. In this example, pouch 107 has two compartments—one compartment securing pressure board 430 and one compartment securing bladder 110 and smoothing layer 440 (illustrated in FIG. 5 but optional—smoothing layer 440 may be the outside of pouch 107 that is between bladder 110 and the patient). Pouch 107 may have a dedicated hole through which right-angle connector 115 and reinforced sleeve 120 connect to tube 125, or a divider within pouch 107 that separates bladder 110 from pressure board 430 may extend only partially through pouch 107.

A next layer after pouch 107 may be bladder 110. Between bladder 110 and the patient is a smoothing layer. In one aspect, smoothing layer 440 is the next layer and within pouch 107, as a distinct and separate component. In one aspect, the last layer of pouch 107 is the smoothing layer and there is no separate, distinct component (i.e. smoothing layer 440 is replaced by an outside layer of pouch 107).

Aspects of systems 100 are described with respect to a pouch, a wrap, double wrap, binders, etc. In one aspect, post-surgical compression garments, for example those provided by Marena Group LLC, Macom Medical and Contour MD, aid in recovery by providing static compression. The garments may cover some or all the torso, legs, buttocks, arms, chin/neck/head, and a combination of the above. The garments are sized for various body types and stages of recovery, from immediate post-surgery to several months out. In one aspect, the pouch in any of post-operative therapeutic pressure systems 100 may be integrated into a post-surgical compression garment. In one aspect, pouch 110 as recited herein is an integral part of a post-surgical garment and all aspects of pouch 110 may be contained or available in the garment. One feature of a garment is a circumferential continuity of material that provides a static pressure to an extremity, torso, or head/neck/chin area.

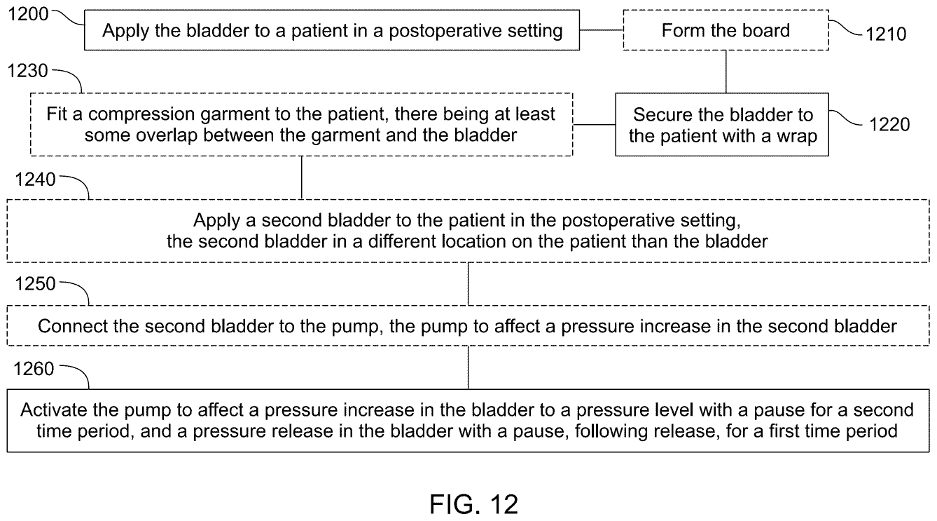

In one aspect, a compression garment covering at least the abdomen and lower back may have two sets of pockets, one set adjacent to the abdomen (front) and the second adjacent to the lower back (rear). Each set of pockets may have an inner pocket and an outer pocket. A bladder and smoothing layer may be placed in the inner pocket. In one aspect, a bladder and smoothing layer may be in either one or both of the front pocket and rear pockets. A pressure board may not be needed immediately post-surgery. At an appropriate stage of recovery and as tolerated by the patient, the pressure board may be added to one of the outer pockets in either the front, rear, or both sets of pockets. In this aspect, more than one bladder may be used in one of systems 100 (see description for FIG. 12). Additional wraps or binders may be placed on top of either the garment/bladder/smoothing layer combination, or on top of the pressure board/garment/bladder/smoothing layer combination. One of skill in the art will recognize that the above application of systems 100 to compression garments for the torso and back also applies to the legs, buttocks, chest, arms, and chin/neck/head. Multiple bladders may be used, for example on the legs, a leg and an arm, chest and back, etc.

Systems 100 may be a postoperative rhythmic compression garment, the garment comprising: an inflatable bladder, the bladder pressure-limited to a peak pressure of 25 mmHg and having an uninflated surface area no greater than 260 square inches; a pump coupled to the bladder, the pump configured to maintain a plurality of positive, non-zero pressures within the bladder; an (optional) pressure board adjacent to the bladder, the board resistant to deformation; a circumferential flexible material configured to receive and secure the pressure board and the bladder, the bladder further having two sides, the pressure board immediately adjacent to one of the two sides; and a smoothing layer immediately adjacent to the bladder on the side opposite the pressure board.

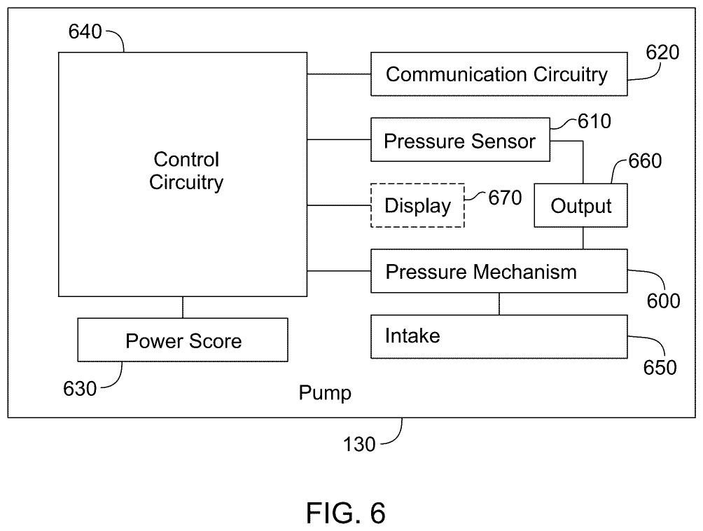

FIG. 6 is a schematic diagram of one aspect of a pump for a postoperative therapeutic pressure system. In one aspect, pump 130 may be fluidly coupled to bladder 110 through tube 125, and pump 130 is configured to monitor and adjust the air pressure of bladder 110 as described herein.

In one aspect, pump 130 may include pressure mechanism 600, pressure sensor 610, communication circuitry 620, power source 630, control circuitry 640, intake 650, output 660 and optionally display 670. Output 660 may provide a fluid connection between pressure mechanism 600 and tube 125, for example. In some aspects, pump 130 may include other types of sensor interface, for example for heart rate, blood pressure, glucose levels, oxygen saturation, etc.

In various aspects, pressure mechanism 600 may be in fluid communication with bladder 110 and configured to inflate or deflate bladder 110 to adjust the air pressure of bladder 110. In some aspects, for example, pressure mechanism 600 may include a pump body (e.g., a cylinder, a casing) configured to hold a volume of fluid (e.g., air). In some aspects, pressure mechanism 600 may include a displacement member disposed in the pump body and configured to reciprocate or rotate upon the application of rotary movement to draw air in through intake 650 and displace a bolus of air into or out of the interior of bladder 110. The displacement member may include a diaphragm, a piston, or an impeller. In some aspects, pressure mechanism 600 may include a motor coupled to the pump body and configured to drive rotation or reciprocation of the displacement member such that displacement member displaces the bolus of air in or out of the interior of inflatable bladder 110. The motor may be configured to reverse rotary or reciprocating movement of displacement member so that pressure mechanism 600 may either inflate or deflate bladder 110. In some aspects, the motor may include, for example, a micro-motor, an induction motor, a synchronous motor, a brush DC motor, a brushless motor, a variable reluctance motor, a permanent magnet motor, or a piezoelectric motor.

Pressure mechanism 600 may connect to tube 125 through output 660 of pump 130 to inflatable bladder 110 so that air may pass from pressure mechanism 600 to the interior of bladder 110. In some aspects, pressure mechanism 600 may include a pump valve configured to be set in a closed position when pressure mechanism 600 is not activated to prevent fluid communication between pressure mechanism 600 and the interior of bladder 110, in order to hold pressure in bladder 110. In some aspects, the pump valve may be configured to switch from a closed position to an open position when pressure mechanism 600 is activated to permit fluid communication between pressure mechanism 600 and the interior of bladder 110.

In some aspects, pressure sensor 610 may be operatively connected to control circuitry 640 (e.g., directly connected or through electrical wiring) and configured to measure the air pressure of bladder 110 through the fluid connection provided by output 660 and tube 125. In some aspects, pressure sensor 610 may be configured to transmit a measurement signal representing the measured air pressure of bladder 110 to control circuitry 640. In some aspects, pressure sensor 610 may be configured to sample air pressure measurements of bladder 110 often enough (e.g., at least once every second) to indicate how the air pressure of bladder 110 is changing while being used by the patient or while being adjusted by pressure mechanism 600.

In some aspects, pressure sensor 610 may be an absolute pressure sensor, a gauge pressure sensor, or a differential pressure sensor. In some aspects, pressure sensor 610 may measure air pressure through electrical resistance (e.g., using a strain-gauge coupled to a resistor to measure pressure changes by change in resistance), capacitance (e.g., using a pair of flexible plates separated by a dielectric to measure pressure changes by change in capacitance), or inductance (e.g., using a diaphragm coupled to a metal core to measure pressure changes by change in induced current).

In various aspects, communication circuitry 620 is operatively linked to control circuitry 640 and configured to electronically communicate (e.g., via a wireless and/or wired connection) data and power with an external device (e.g., charging pad, a mobile device). In some aspects, communication circuitry 620 may include an inductor configured to generate a current from an applied magnetic field to power control circuitry 640 or communicate information. In some aspects, the inductor of communication circuitry 620 may generate a current or communicate information by using Near Field Communication (NFC). In some aspects, the inductor of the communication circuitry 620 may comprise a metal coil, such as, for example, a copper coil. In some aspects, communication circuitry 620 may include an antenna configured to receive and transmit radio frequency signals with an external device (e.g., a mobile device) according to various wireless communication protocols, such as a short-range wireless technology standard, such as an Advanced and Adaptive Network Technology (ANT™) standard, a BLUETOOTH® or A BLUETOOTH LOW ENERGY® (BLE) standard (e.g., BLE 4.0). In some aspects, the antenna of communication circuitry 620 may be a ceramic chip antenna, a metal plate antenna, a microstrip patch antenna, a planar Inverted-F antenna, a printed antenna, or a flexible printed antenna. In some aspects, communication circuitry 620 may include any type of circuitry components, such as amplifiers, capacitors, voltage regulators, rectifiers, etc., suitable for amplifying, filtering, and regulating signals received or transmitted by the inductor or the antenna.

In some aspects, power source 630 is operatively connected to pressure mechanism 600 through control circuitry 640 and configured to store electrical energy and transmit power to pressure mechanism 600 and control circuitry 640. In some aspects, power source 630 may include a rechargeable battery (e.g., lithium-ion battery). In some aspects, power source 630 may include an input interface, such as, for example, a USB cable connector, for receiving current from an external power source. In some aspects, power source 630 may be recharged by receiving a current generated by the inductor of communication circuitry 620 so that power source 630 may be recharged by a remote device (e.g., a charging pad).

In some aspects, optional display 670 may be operatively linked to control circuitry 640 and disposed on any portion of pump 130 to indicate a status of the postoperative therapeutic pressure systems 100. In some aspects, display 670 is configured to illuminate when pressure mechanism 600 is activated to indicate that pressure mechanism 600 is adjusting the air pressure of bladder 110. In some aspects, display 670 may include light emitting diodes (LED) disposed on pump 130. In some aspects, the LED of optional display 670 may be configured to indicate an air pressure measurement of bladder 110.

In various aspects, control circuitry 640 may include an integrated circuit (e.g., an application specific integrated circuit) operatively linked to various elements of pump 130, such as pressure mechanism 600, pressure sensor 610, communication circuitry 620, power source 630, and optional display 670, to monitor and control operations of postoperative therapeutic pressure systems 100. In some aspects, control circuitry 640 may include a semiconductor substrate (e.g., printed circuit board) and analog and/or digital circuitry components fabricated in the semiconductor substrate. In some aspects, pressure mechanism 600, pressure sensor 610, and communication circuitry 620 may be directly coupled to semiconductor substrate of control circuitry 640.

In some aspects, control circuitry 640 may include a processor (e.g., a microprocessor, a multi-core processor, a central processing unit) configured to receive signals transmitted from pressure sensor 610 and communication circuitry 620 as inputs and generate actuation signals transmitted to pressure mechanism 600 for adjusting the air pressure of bladder 110 and display 670 for indicating status of postoperative therapeutic pressure systems 100. In some aspects, control circuitry 640 may include electronic inputs for receiving signals transmitted from pressure sensor 610 and communication circuitry 620. In some aspects, control circuitry 640 may include electrical outputs and actuator circuitry, such as amplifiers, to generate and drive actuator signals to pressure mechanism 600 and display 670. Control circuitry 640 includes a clock, for example some type of oscillator by which control circuitry 640 tracks the passage of time.

In some aspects, control circuitry 640 may include memory comprising computer storage media in the form of volatile memory, such as random access memory (RAM), and/or nonvolatile memory, such as read-only memory (ROM). In some aspects, the memory of control circuitry 640 may be configured to store computer readable instructions, data structures, program modules, and other data, which are inputted to the processor for the execution of operations, as described herein. In some aspects, control circuitry 640 may include any type of circuitry components, such as a bus, for transmitting instructions stored in the memory to the processor.

In operation, control circuitry 640 may be configured to automatically adjust the air pressure of inflatable bladder 110 in response to receiving inputs from at least pressure sensor 610, and command signals from a remote device, for example signals received through communication circuitry 620. In some aspects, control circuitry 640 is configured to receive a measurement signal representing the measured air pressure of bladder 110. Upon receiving a measurement signal from pressure sensor 610, control circuitry 640 may be configured to determine if the measured air pressure of bladder 110 is not exceeding the peak pressure, and therefore maintaining the pressure limit in bladder 110. In one aspect, the peak pressure may be set to 25 mmHg, allowing a range in pressure from zero to 25 mmHG. In one aspect, the peak pressure may be set to 50 mmHg, allowing a range in pressure from zero to 50 mmHG. In one aspect, the peak pressure may be set to 75 mmHg, allowing a range in pressure from zero to 75 mmHG. Other peak pressures may be established, for example 100 mmHg.

In one aspect, if pump 130 through control circuitry 640 determines that measured air pressure exceeds the peak pressure, then control circuitry 640 is configured to transmit a signal to pressure mechanism 600 such that pressure mechanism 600 adjusts the air pressure of bladder 110 based on a difference between the measured air pressure and the peak pressure.

In some aspects, control circuitry 640 is configured to have a rapid release mode, in which control circuitry 640 actuates pressure mechanism 600 to deflate bladder 110 to a minimal (for example, zero mmHg of pressure over the ambient air pressure) to provide therapeutic pressure massage to the patient in conjunction with an increase in pressure in bladder 110.

Optionally, a remote device may be used to communicate with pump 130 through communication circuitry 620 to monitor and regulate the air pressure of bladder 110. The optional remote device may comprise a smartphone, a tablet, a near field communication device, a short-range wireless technology standard device, such as a BLUETOOTH® device, a radio frequency identification (RFID) device, a desktop computer, a smartwatch, or other suitable device.

Operation of postoperative therapeutic pressure systems 100 as described above may be further described in the following Figures, with reference made back to FIG. 6.

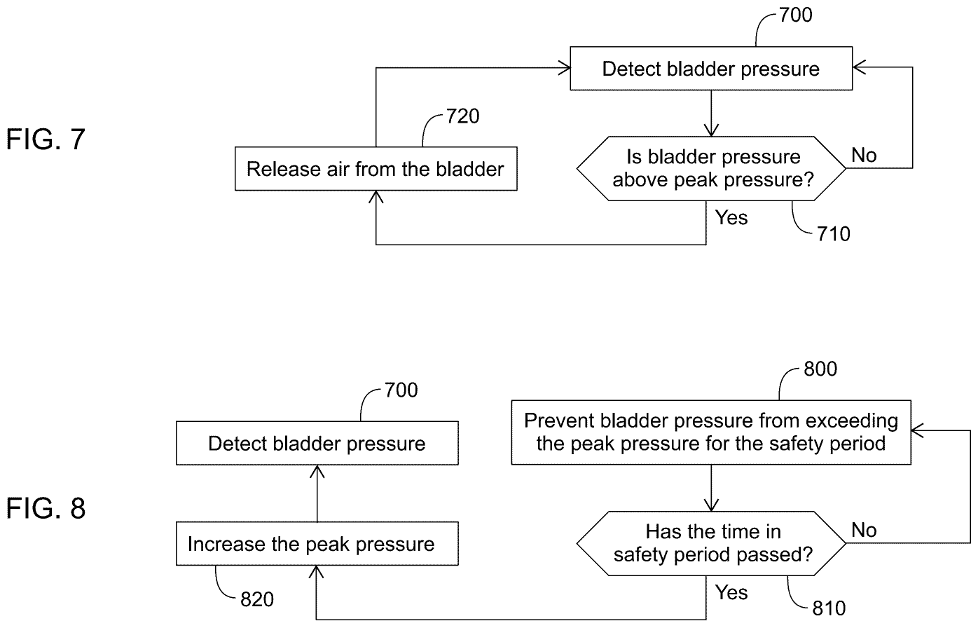

FIG. 7 is a block diagram illustrating a peak pressure limit for a bladder pressure in a postoperative therapeutic pressure system. Peak pressure in postoperative therapeutic pressure systems 100 (systems 100) may vary, for example by the amount of time a patient is post-surgery. Peak pressure may initially be 25 mmHg, and over time increase to 50, 70, or higher pressure, for example 100 mmHg. In one aspect, increases in peak pressure may be pre-programmed into pump 130. In one aspect, increases in peak pressure may be made in pump 130 by a healthcare practitioner according to patient needs. One of skill in the art will recognize that peak pressure may be set to a specific number that is within a range, for example from 20-30 mmHg, from 40-50 mmHg, from 60-70 mmHg and from 90-100 mmHg. In one aspect, peak pressure may be set to a pressure that has therapeutic effect, from 5-100 mmHg.

In block 700, pump 130 through pressure sensor 610 may detect the bladder pressure. Control circuitry 640 compares, in block 710, the detected bladder pressure with the peak pressure. If the detected bladder pressure is above the peak pressure then in block 720, pump 130 releases air from bladder 110. After releasing air from bladder 110, pump 130 detects bladder pressure in block 700. A sample rate of once per second may be used, for example. When the detected bladder pressure is not above the peak pressure, pump 130 may detect bladder pressure at the sample rate.

FIG. 8 is a block diagram illustrating a pressure limit for a safety period in a postoperative therapeutic pressure system. A safety time period, measured in hours after surgery, may be defined in which bladder pressure should not exceed a medically determined pressure level. In one aspect, the safety period may be 7 days for a peak pressure of 25 mmHg. For positive patient outcomes, pump 130 prevents pressure in bladder 110 from exceeding the peak pressure for a safety time period, in block 800. Control circuitry 640 checks whether the elapsed time is within the safety period, in block 810. If the elapsed time is within the safety period, then control circuitry 640 prevents pressure in bladder 110 from exceeding the peak pressure. Control circuitry 640 may check the elapsed time every minute, or every second, or at some other sample rate. If the elapsed time is outside the safety period, then in block 820 the peak pressure may increase. In one aspect, an increase in peak pressure may be programmed into control circuitry 640. In one aspect, the pressure may increase as a result of input by a healthcare provider.

Following an increase in pressure from block 820, systems 100 may detect the pressure in bladder 110, in block 700 (also see FIG. 7).

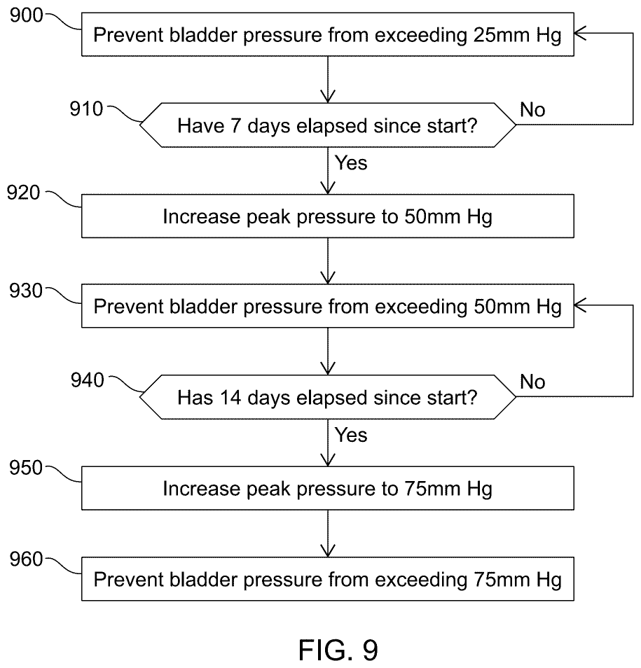

FIG. 9 is a block diagram illustrating an increase in peak pressure in a postoperative therapeutic pressure system. In one aspect, FIG. 9 represents peak pressure increases and timeframes for a particular patient profile. Other patient profiles that include different peak pressures than those listed, or different timeframes than those listed, or both, are possible.

In block 900, pump 130, through pressure mechanism 600, pressure sensor 610, output 660 and control circuitry 640, prevents the pressure in bladder 110 from exceeding 25 mmHg. Assuming an initial starting time for one of systems 100 in a postoperative setting, control circuitry 640 checks whether 7 days have passed since starting, in block 910. Pump 130 continues preventing the pressure in bladder 110 from exceeding 25 mmHG until 7 days have elapsed.

Once 7 days have elapsed, in block 920 pump 130 may increase peak pressure to 50 mmHg. In block 930, pump 130, through pressure mechanism 600, pressure sensor 610, output 660 and control circuitry 640, prevents the pressure in bladder 110 from exceeding 50 mmHg. Control circuitry 640 checks whether 14 days have passed since starting, in block 940. Pump 130 continues preventing the pressure in bladder 110 from exceeding 50 mmHG until 14 days have elapsed.

Once 14 days have elapsed, in block 950 pump 130 may increase peak pressure to 75 mmHg. In block 960, pump 130, through pressure mechanism 600, pressure sensor 610, output 660 and control circuitry 640, prevents the pressure in bladder 110 from exceeding 75 mmHg.

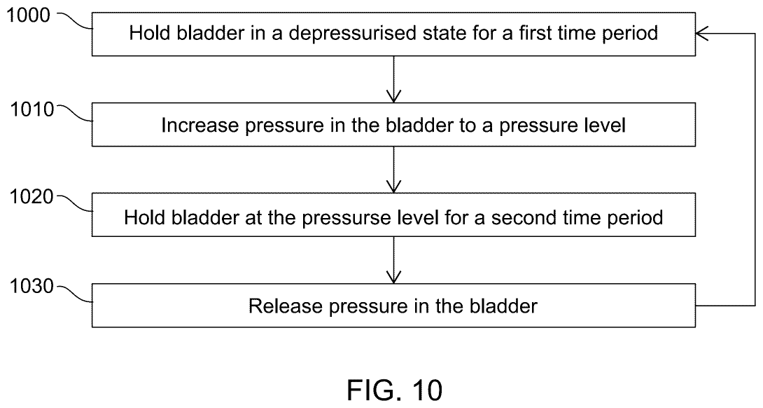

FIG. 10 is a block diagram illustrating rhythmic compressions in a postoperative therapeutic pressure system. As previously described, rhythmic compression is one mechanism by which lymphatic massage may reduce edema after surgical procedures. Postoperative therapeutic pressure systems 100 use the concept of Rhythmic Compression Therapy (RCT) to mechanically “pump” postoperative edema out of the skin and soft tissue and into the lymphatic system for clearance. One aspect of rhythmic compression is illustrated in FIG. 10.

In block 1000, pump 130 may keep bladder 110 in a depressurized state for a certain period of time. This first time period may be set based on a variety of factors, for example stage of patient healing, time elapsed since surgery, what part of the patient's body was operated upon, proximity of incisions, etc. An amount of time in the first time period may be from 10 seconds to 2 minutes, or from 10 seconds 70 seconds, or from 10 seconds to 20 seconds, or from 25 seconds to 35 seconds, or from 50 seconds to 70 seconds, or 25 seconds.

In block 1010, pump 130, implementing intake 650, pressure mechanism 600, output 660, pressure sensor 610 and control circuitry 640, increases pressure in bladder 110 to a pressure level. The pressure level to which bladder 110 is raised may depend on the peak pressure and may be from 5 mmHg up to 30 mmHg, or from 20 mmHg to 30 mmHg, or from 40 mmHg to 60 mmHg, or from 70 mmHg to 80 mmHg, or 25 mmHg, or 50 mmHg, or 75 mmHg. Pump 130 may increase pressure in block 1010 up to, but not exceeding, the peak pressure. In one aspect, pump 130 may pressurize bladder 110 up to peak pressure. In one aspect, pump 130 may pressurize bladder 110 to a pressure level below peak pressure. One reason that pump 130 may pressurize bladder 110 to below-peak-pressure is for patient comfort. In one aspect, bladder 110 with no additional air may be at (or above) peak pressure, for example when initially applied or later, due to swelling. If bladder 110 is at, or above, peak pressure without additional air, then additional air may not be added to bladder 110 until the pressure is below peak pressure.

In block 1020, pump 130 holds the pressure in bladder 130 at the pressure level for a certain period of time. This second time period may be set based on a variety of factors, for example stage of patient healing, time elapsed since surgery, what part of the patient's body was operated upon, proximity of incisions, etc.

Block 1030 shows pump 130 releasing pressure in bladder 110 before repeating the cycle with block 1000. In one aspect, the rhythmic compression described with FIG. 10 may continue for minutes, hours or days depending on the needs of the patient. The rhythmic compression can be halted and started, also based on the needs of the patient. Between usages of one of systems 100, peak pressure and time periods may be adjusted, either manually by healthcare provider (or by the patient), or by virtue of pump programming.

In one aspect of systems 100, after block 1020, instead of releasing pressure, pump 130 may increase pressure, followed by a pause. In a stepwise, incremental manner, pump 130 may increase pressure and hold, up to peak pressure, before releasing. FIG. 10 illustrates a single step, however one of skill in art will recognize that multiple steps are possible. For example, there may be 2 steps, or 3, 4, 5, 6, 7, 8, 9, 10 or more steps. Each step, or increase in pressure, may be approximately the same (for example, a 10 mmHg increase in each step results in a first pressure level at 10 mmHg, a second pressure level at 20 mmHg, a third pressure level at 30 mmHg, etc.), or the steps may differ in the amount of pressure increase (for example, an increase of 20 mmHg (20 mmHg total pressure), followed by an increase of 15 mmHg (35 mmHg total pressure), followed by 10 mmHg (45 mmHg total pressure), etc.). In one aspect, the hold time between steps is the same. In one aspect, the hold time between steps differs.

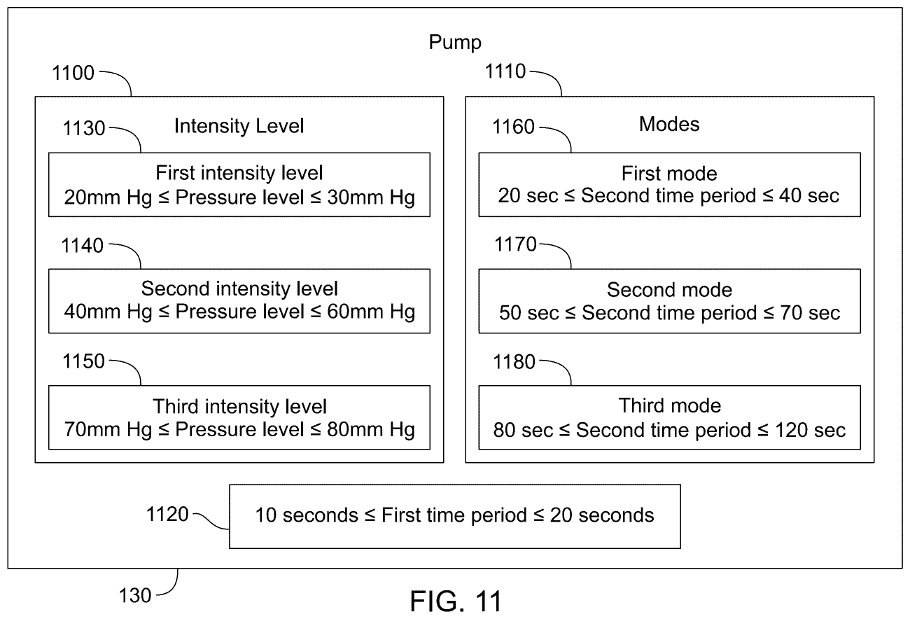

FIG. 11 is a block diagram illustrating modes and intensity levels for one aspect of a pump in a postoperative therapeutic pressure system. FIG. 11 illustrates one aspect of rhythmic compressions for pump 130. FIG. 11 may be understood in conjunction with FIG. 10, in one aspect references in FIG. 10 to pressure level, first time period and second time period. In one aspect, pump 130 may be programmed with or capable of multiple intensity levels, block 1100, multiple modes, block 1110, and at least a first time period from 10 to 20 seconds, block 1120. Intensity levels refer to pressure in bladder 110 during lymphatic massage (rhythmic compressions). Bladder 110 is pressure-limited to the peak pressure (and subject to the safety period) regardless of intensity level, i.e. pump 130 will not operate at an intensity level that would provide pressure higher than the peak pressure. Modes refer to the amount of time a given pressure level is held during lymphatic massage (rhythmic compressions). In a situation where pressure in bladder 110 is measured above peak pressure, one of systems 100 may not perform any rhythmic massage until the pressure in bladder is below peak pressure.

Block 1100 includes multiple intensity levels. In one aspect, a first intensity level is illustrated with the pressure level from 20 to 30 mmHg, block 1130. In one aspect, a second intensity level is illustrated with the pressure level from 40 to 60 mmHg, block 1140. In one aspect, a third intensity level is illustrated with the pressure level from 70 to 80 mmHg, block 1150.

Block 1110 includes multiple modes. In one aspect, a first mode is illustrated with the second time period from 20 to 40 seconds, block 1160. In one aspect, a second mode is illustrated with the second time period from 50 to 70 seconds, block 1170. In one aspect, a third mode is illustrated with the second time period from 80 to 120 seconds, block 1180.

In operation and with reference to FIGS. 10 and 11, pump 130 may function as follows. In one aspect, with first intensity level, block 1130 and first mode, 1160, pump 130 may hold bladder 110 in a depressurized state for 10 to 20 seconds (first time period, block 1120). Following the first time period, pump 130 may increase the pressure in bladder 110 to a pressure level from 20 to 30 mmHg and hold it there for 20 to 40 seconds.

In one aspect, with first intensity level, block 1130 and second mode, 1170, pump 130 may hold bladder 110 in a depressurized state for 10 to 20 seconds (first time period, block 1120). Following the first time period, pump 130 may increase the pressure in bladder 110 to a pressure level from 20 to 30 mmHg and hold it there for 50 to 70 seconds.

In one aspect, with first intensity level, block 1130 and third mode, 1180, pump 130 may hold bladder 110 in a depressurized state for 10 to 20 seconds (first time period, block 1120). Following the first time period, pump 130 may increase the pressure in bladder 110 to a pressure level from 20 to 30 mmHg and hold it there for 80 to 120 seconds.

In one aspect, with second intensity level, block 1140 and first mode, 1160, pump 130 may hold bladder 110 in a depressurized state for 10 to 20 seconds (first time period, block 1120). Following the first time period, pump 130 may increase the pressure in bladder 110 to a pressure level from 40 to 60 mmHg and hold it there for 20 to 40 seconds.

In one aspect, with second intensity level, block 1140 and second mode, 1170, pump 130 may hold bladder 110 in a depressurized state for 10 to 20 seconds (first time period, block 1120). Following the first time period, pump 130 may increase the pressure in bladder 110 to a pressure level from 40 to 60 mmHg and hold it there for 50 to 70 seconds.

In one aspect, with second intensity level, block 1140 and third mode, 1180, pump 130 may hold bladder 110 in a depressurized state for 10 to 20 seconds (first time period, block 1120). Following the first time period, pump 130 may increase the pressure in bladder 110 to a pressure level from 40 to 60 mmHg and hold it there for 80 to 120 seconds.

In one aspect, with third intensity level, block 1150 and first mode, 1160, pump 130 may hold bladder 110 in a depressurized state for 10 to 20 seconds (first time period, block 1120). Following the first time period, pump 130 may increase the pressure in bladder 110 to a pressure level from 70 to 80 mmHg and hold it there for 20 to 40 seconds.

In one aspect, with third intensity level, block 1150 and second mode, 1170, pump 130 may hold bladder 110 in a depressurized state for 10 to 20 seconds (first time period, block 1120). Following the first time period, pump 130 may increase the pressure in bladder 110 to a pressure level from 70 to 80 mmHg and hold it there for 50 to 70 seconds.

In one aspect, with third intensity level, block 1150 and third mode, 1180, pump 130 may hold bladder 110 in a depressurized state for 10 to 20 seconds (first time period, block 1120). Following the first time period, pump 130 may increase the pressure in bladder 110 to a pressure level from 70 to 80 mmHg and hold it there for 80 to 120 seconds.

Pump 130 may repeat the above recited rhythmic compressions in any of systems 100 as many times as needed for a patient. One of skill in the art will recognize that other time periods and pressures are possible, and the above description is exemplary.

FIG. 12 is a block diagram illustrating one method for providing postoperative lymphatic massage with a postoperative therapeutic pressure system. The blocks in FIG. 12 are not necessarily applied in the order listed, and not all of the blocks are needed for postoperative lymphatic massage (rhythmic compressions). The blocks in FIG. 12 are given with reference to systems 100 as described herein and FIGS. 1-11.

In block 1200, apply the bladder to a patient in a postoperative setting. In optional block 1210, form the (pressure) board. The pressure board may be introduced early in the healing process, or later when higher pressure is applied. In block 1220, secure the bladder to the patient with a wrap. In optional block 1230, fit a compression garment to the patient, there being at least some overlap between the garment and the bladder. In optional block 1240, apply a second bladder to the patient in the postoperative setting, the second bladder in a different location on the patient than the bladder. In optional block 1250, connect the second bladder to the pump, the pump to affect a pressure increase in the second bladder. In one aspect, a single output from the pump may be split between the two bladders. In one aspect, the pump may have separate output, pressure sensor and pressure mechanisms for each bladder to independently manage the two bladders. In one aspect, there is a bladder on the torso and a bladder on an extremity. In one aspect, there is a bladder on each of an extremity. More than two bladders may be used if needed. In block 1260, activate the pump to affect a pressure increase in the bladder to a pressure level with a pause for a second time period, and a pressure release in the bladder with a pause, following release, for a first time period.

In one aspect, two or more bladders may be placed in a single pouch. In one aspect, multiple bladders may be placed in the same compartment of a single pouch, or in separate compartments. In one example with a garment replacing the pouch, or having a pouch (or multiple pouches) integrated into the garment, multiple bladders may be positioned as needed in that garment. Multiple bladders along an extremity, for example, may operate cooperatively to sequentially pulse pressure along the extremity from distal to proximal. One effect of this may be to encourage movement of excess fluid from the extremity to a patient's core. In one aspect, this may be implemented on a patient's torso, encouraging movement of fluid from a patient's upper abdomen toward lower abdomen, for example. One of skill in the art will recognize that other configurations and combinations are possible.

The aspects and features mentioned and described together with one or more of the previously detailed examples and figures, may as well be combined with one or more of the other examples in order to replace a like feature of the other example or in order to additionally introduce the feature to the other example.

Examples may further be or relate to a computer program having a program code for performing one or more of the above methods, when the computer program is executed on a computer or processor. Steps, operations or processes of various above-described methods may be performed by programmed computers or processors. Examples may also cover program storage devices such as digital data storage media, which are machine, processor or computer readable and encode machine-executable, processor-executable or computer-executable programs of instructions. The instructions perform or cause performing some or all the acts of the above-described methods. The program storage devices may comprise or be, for instance, digital memories, magnetic storage media such as magnetic disks and magnetic tapes, hard drives, or optically readable digital data storage media. Further examples may also cover computers, processors or control units programmed to perform the acts of the above-described methods or (field) programmable logic arrays ((F) PLAs) or (field) programmable gate arrays ((F) PGAs), programmed to perform the acts of the above-described methods.

The description and drawings merely illustrate the principles of the disclosure. Furthermore, all examples recited herein are principally intended expressly to be only for pedagogical purposes to aid the reader in understanding the principles of the disclosure and the concepts contributed by the inventor(s) to furthering the art. All statements herein reciting principles, aspects, and examples of the disclosure, as well as specific examples thereof, are intended to encompass equivalents thereof.

A functional block denoted as “means for . . . ” performing a certain function may refer to a circuit that is configured to perform a certain function. Hence, a “means for something” may be implemented as a “means configured to or suited for something”, such as a device or a circuit configured to or suited for the respective task.

Functions of various elements shown in the figures, including any functional blocks labeled as “means”, “means for providing a sensor signal”, “means for generating a transmit signal.”, etc., may be implemented in the form of dedicated hardware, such as “a signal provider”, “a signal processing unit”, “a processor”, “a controller”, etc. as well as hardware capable of executing software in association with appropriate software. When provided by a processor, the functions may be provided by a single dedicated processor, by a single shared processor, or by a plurality of individual processors, some of which or all of which may be shared. However, the term “processor” or “controller” is by far not limited to hardware exclusively capable of executing software but may include digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included.

A block diagram may, for instance, illustrate a high-level circuit diagram implementing the principles of the disclosure. Similarly, a flow chart, a flow diagram, a state transition diagram, a pseudo code, and the like may represent various processes, operations or steps, which may, for instance, be substantially represented in computer readable medium and so executed by a computer or processor, whether or not such computer or processor is explicitly shown. Methods disclosed in the specification or in the claims may be implemented by a device having means for performing each of the respective acts of these methods. In block diagrams, blocks with dashed lines may be optional elements, actions, configurations, etc.

It is to be understood that the disclosure of multiple acts, processes, operations, steps, or functions disclosed in the specification or claims may not be construed as to be within the specific order, unless explicitly or implicitly stated otherwise, for instance for technical reasons. Therefore, the disclosure of multiple acts or functions will not limit these to a particular order unless such acts or functions are not interchangeable for technical reasons. Furthermore, in some examples a single act, function, process, operation or step may include or may be broken into multiple sub-acts, -functions, -processes, -operations or -steps, respectively. Such sub acts may be included and part of the disclosure of this single act unless explicitly excluded.

Furthermore, the following claims are hereby incorporated into the detailed description, where each claim may stand on its own as a separate example. While each claim may stand on its own as a separate example, it is to be noted that—although a dependent claim may refer in the claims to a specific combination with one or more other claims—other examples may also include a combination of the dependent claim with the subject matter of each other dependent or independent claim. Such combinations are explicitly proposed herein unless it is stated that a specific combination is not intended. Furthermore, it is intended to also include features of a claim to any other independent claim even if this claim is not directly made dependent on the independent claim.

What is claimed is:

1. A postoperative therapeutic pressure system, the system comprising:
an inflatable bladder, the bladder pressure-limited to a peak pressure of 25 mmHg and having an uninflated surface area no greater than 260 square inches;
a pump coupled to the bladder, the pump configured to maintain a plurality of positive, non-zero pressures within the bladder;
a pressure board adjacent to the bladder, the board resistant to deformation;
a pouch configured to receive and secure the pressure board and the bladder, the bladder further having two sides, the pressure board immediately adjacent to one of the two sides; and
a smoothing layer immediately adjacent to the bladder on the side opposite the pressure board.

2. The system of claim 1 further comprising:
a pressure sensor in the pump configured to provide the pressure for the bladder, the pump further configured to maintain the pressure-limit in the bladder.

3. The system of claim 2, the pump further configured to increase the peak pressure after a safety period.

4. The system of claim 3, wherein the safety period is 7 days.

5. The system of claim 2, the pump further configured to increase the peak pressure to 50 mmHg after 7 days, and to increase the peak pressure to 75 mmHg after 14 days.

6. The system of claim 3, the pump further configured, with the bladder in a depressurized state, to hold for a first time period, to increase pressure in the bladder to a pressure level and pause for a second time period, and release pressure in the bladder.

7. The system of claim 6, the pressure board further comprising a moldable, foam-aluminum composite.

8. The system of claim 6, the pump further configured with a first intensity level, a second intensity level and a third intensity level, the first intensity level having a pressure level from 20 to 30 mmHg, the second intensity level having a pressure level from 40 to 60 mmHg, the third intensity level having a pressure level from 70 to 80 mmHg, the pump further configured to maintain bladder pressure at or under the peak pressure.

9. The system of claim 8, wherein the first time period is from 10 to 20 seconds, the pump further configured with a first mode, a second mode and a third mode, the first mode having a second time period from 20 to 40 seconds, the second mode having a second time period from 50 to 70 seconds, and the third mode having a second time period from 80 to 120 seconds.

10. The system of claim 6, wherein the pressure board, the bladder and the smoothing layer have the same shape.

11. The system of claim 10 further comprising:
a multi-panel, stretchable and compressive wrap coupled to the pouch, the pouch configured to be removably affixed to the wrap.

12. The system of claim 1, the pouch being neoprene rubber with a plurality of layers forming pockets, the pockets having VELCRO enclosures, the smoothing layer being one of the layers of the pouch.

13. The system of claim 1, the pouch being neoprene rubber with a plurality of pockets, the pockets having VELCRO enclosures, the smoothing layer being foam and immediately adjacent to the bladder, both the bladder and the smoothing layer in one of the plurality of pockets.

14. The system of claim 13, the pressure board in one of the plurality of pockets not containing the bladder.

15. The system of claim 1 further comprising:
a tube coupled to the pump;
a right-angle connector connected to the bladder; and
a reinforced sleeve between the tube and the right-angle connector.

16. A method for providing postoperative lymphatic massage in a system having an inflatable bladder, the bladder pressure-limited to a peak pressure of 25 mmHg and having an uninflated surface area no greater than 260 square inches, a pump coupled to the bladder, the pump configured to maintain a plurality of positive, non-zero pressures within the bladder, a pressure board adjacent to the bladder, the board being formable and resistant to deformation, a smoothing layer coupled to and adjacent to the bladder, a multi-panel, stretchable and compressive wrap coupled to the bladder, the bladder configured to be removably affixed to the wrap, the method comprising:
applying the bladder to a patient in a postoperative setting;
securing the bladder to the patient with the wrap; and
activating the pump to affect a pressure increase in the bladder to a pressure level with a pause for a second time period, and a pressure release in the bladder with a pause, following release, for a first time period.

17. The method of claim 16 further comprising:
fitting a compression garment to the patient, there being at least some overlap between the garment and the bladder.

18. The method of claim 16, wherein the pressure level is within the range of 5 to 25 mmHg, the first time period from 10 to 20 seconds, the second time period from 30 to 90 seconds.

19. The method of claim 18 further comprising:
forming the board.

20. The method of claim 16 further comprising:
applying a second bladder to the patient in the postoperative setting, the second bladder in a different location on the patient than the bladder; and
connecting the second bladder to the pump, the pump to affect a pressure increase in the second bladder.
